(19) United States
(12) Patent Application Publication
(10) Pub. No.: US 2023/0072046 A1
(43) Pub. Date: Mar. 9, 2023

(54) MEDICAL INSTRUMENT FOR DISLODGING VERTEBRAL DISC MATERIAL FROM A VERTEBRAL DISC IN A VERTEBRAL DISC SPACE VIA A CANNULA

(71) Applicant: Life Spine, Inc., Huntley, IL (US)

(72) Inventor: Daniel P. Predick, Wheat Ridge, CO (US)

(73) Assignee: Life Spine, Inc., Huntley, IL (US)

(21) Appl. No.: 17/895,215

(22) Filed: Aug. 25, 2022

Related U.S. Application Data
(60) Provisional application No. 63/241,145, filed on Sep. 7, 2021.

(51) Int. Cl. A61B 17/32

(52) U.S. Cl. CPC A61B 17/32002; A61B 2017/00261

(57) ABSTRACT

A medical instrument for dislodging vertebral disc material from a vertebral disc in a vertebral disc space via a cannula has a pivoting scoop structure with a scoop that provides a 180° pivot sweep outside of the diameter of the cannula. The medical instrument has an elongate body defining a longitudinal axis with the pivoting scoop structure situated at a distal end of the elongate body. The elongate body has a first elongate shaft and a second elongate shaft situated on and configured for longitudinal axial translation relative to the first elongate shaft. Axial movement of the second elongate shaft controllably swivels the pivoting scoop structure from, through, and between 0° to 180° relative to the longitudinal axis of the elongate body. The scoop structure may also allow for collection and removal of dislodged vertebral disc material.

### CROSS-REFERENCE TO RELATED APPLICATIONS

This U.S. non-provisional patent application claims the benefit of and/or priority under 35 U.S.C. § 119(e) to U.S. provisional patent application Ser. No. 63/241,145 filed Sep. 7, 2021 titled “180° Sweep Plane Vertebral Disc Material Collector and Remover,” the entire contents of which is specifically incorporated herein by reference.

### FIELD OF THE INVENTION

The present invention relates to instruments for spine procedures and, more particularly, to instruments for dislodging vertebral disc material from a vertebral disc in a vertebral disc space of a spine during a cannulated spine procedure.

### BACKGROUND OF THE INVENTION

Many people contend with spine issues due to age, disease, trauma, congenital, and acquired complications and conditions. While some spine issues can be alleviated without surgery, other spine issues necessitate surgery. Spine surgery may entail obtaining and removing vertebral disc material that is now typically accomplished using a cannula in minimally invasive spine surgery or an endoscope in micro invasive spine surgery. With minimally invasive spine surgery, a cannula is inserted through a small incision proximate a vertebral disc space. With micro invasive surgery, an endoscope is inserted through the small incision proximate the vertebral disc space. After the cannula or endoscope is properly situated, a medical instrument for dislodging, or dislodging, collecting, and removing vertebral disc material is then placed through the cannula/endoscope where vertebral disc material is then dislodged, or dislodged and removed.

While there are medical instruments for dislodging, obtaining, and/or removing vertebral disc material using a cannula in minimally invasive spine surgery or micro invasive spine surgery, they are deficient in various respects. One deficiency is the lack of better access to the vertebral disc space. Another deficiency is the amount of vertebral disc material that can be dislodged, or dislodged and gathered at one time. A further deficiency is the small degree of sweep that can be achieved in one swipe.

In view of the above deficiencies and others not mentioned, it is therefore desirous to have a better medical instrument for dislodging vertebral disc material in a minimally or micro invasive, or other spine procedure that requires vertebral disc material to be removed, or dislodging, collecting, and removing vertebral disc material in a minimally or micro invasive, or other spine procedure that requires vertebral disc material to be removed.

The present medical instrument provides a solution to the above, and more.

### SUMMARY OF THE INVENTION

A medical instrument for dislodging vertebral disc material from a vertebral disc in a vertebral disc space via a cannula has a pivoting scoop assembly that provides a 180° collection sweep of vertebral disc material along a longitudinal plane and outside of the diameter of the cannula. The medical instrument is sized especially for use in minimally invasive spine surgery and micro spine surgery. The pivoting scoop assembly may also provide for collection and removal of the dislodged vertebral disc material.

The medical instrument has an elongate body defining a proximal body end, a distal body end, and a longitudinal axis. The elongate body is characterized by a first elongate shaft defining a proximal first elongate shaft end and a distal first elongate shaft end, and a second elongate shaft defining a proximal second elongate shaft end and a distal second elongate shaft end, the second elongate shaft axially movably held on the first elongate shaft. The pivoting scoop assembly includes a scoop and is rotationally attached to the distal first elongate shaft end of the first elongate shaft. Longitudinal axial translation of the second elongate shaft relative to the first elongate shaft results in swiveling (scooping) motion of the scoop of the pivoting scoop assembly. The swiveling motion of the scoop effects dislodging of vertebral disc material from the vertebral disc. The scoop may also effect collection and removal of the dislodged vertebral disc material via the swiping motion.

Controllable translation of the second elongate shaft (the actuator shaft) relative to the first elongate shaft (the stationary shaft) swivels or pivots the scoop from, through, and between 0° to 180°, relative to the longitudinal axis of the elongate body.

Where the vertebral disc material scoop is configured to dislodge vertebral disc material via a swiping motion for collection and removal by one or more other medical instruments (not shown), the distal first elongated shaft end of the first elongate shaft is bifurcated. The scoop defines a proximal scoop end, a distal scoop end, and is pivotally connected to the bifurcated distal first elongate shaft end via a pivot pin. In particular, the proximal scoop end has a slot in which is received a first portion of a pivot structure that is pivotally coupled to the proximal scoop end via a first pivot structure pin, with a second portion of the pivot structure pivotally coupled to a bifurcated distal second elongate shaft end of the second elongate shaft via a second pivot structure pin. An actuator structure situated proximate the distal second elongate shaft end of the second elongate shaft and the second portion of the pivot structure retains an actuator pin of the first elongate shaft such that axial translation of the second elongate shaft on the first elongate shaft along the longitudinal axis of the elongate body, causes the pivot structure to swivel effecting pivoting of the scoop about the first portion of the pivot structure and the bifurcated distal first elongate shaft end from, through, and between 0° to 180° relative to the longitudinal axis of the elongate body, the actuator pin of the first elongate shaft providing a guide for the actuator structure.

In this form, the actuator structure is a flange proximate the distal second elongate shaft end of the second elongate shaft with a horizontal slot relative to the longitudinal axis of the elongate body that retains the actuator pin of the first elongate shaft for axial movement on and relative to the actuator pin, the flange constrained in a notch in the first elongate shaft proximate the distal first elongate shaft end.

In this form, the second shaft is movably connected to the first elongate shaft via a slot.

In this form, the scoop is fashioned as a scoop arm with a cutout extending through the scoop arm.

In the another form, the scoop of the scoop assembly of the medical instrument is configured to dislodge vertebral disc material, collect the dislodged vertebral disc material via a swiping motion, and capture the collected vertebral disc material within the scoop and against a mating area in the first elongate shaft for removal of the collected vertebral disc material by the medical instrument.

In this form, the scoop is pivotally connected to the bifurcated distal first elongated shaft end of the first elongate shaft via a pivot pin. A bifurcated proximal end of the scoop is coupled to a pivot actuator structure at the distal second elongate shaft end of the second elongate shaft situated between the bifurcated proximal end of the scoop and retains a pin of the scoop such that translation of the second elongate shaft on the first elongate shaft along the longitudinal axis of the elongate body causes the pin of the scoop to move vertically within a slot of the pivot actuator structure, thereby pivoting the scoop about the pivot pin of the bifurcated distal first elongate shaft end of the first elongate shaft from, between, and through 0° to 180° relative to the longitudinal axis of the elongate body.

In this form, the actuator structure is a flange at the distal second elongate shaft end of the second elongate shaft with a vertical slot relative to the longitudinal axis of the elongate body that movably retains the pin of the scoop.

In this form, the second elongate shaft is movably connected to the first elongate shaft via a dovetail configuration.

In this form, the scoop is fashioned as a scoop arm with an inner concave surface for receiving dislodged vertebral disc material.

Further aspects of the present invention will become apparent from consideration of the drawings and the following description of embodiments of the invention. A person skilled in the art will realize that other forms of the invention are possible and that the details of the invention can be modified in a number of respects without departing from the inventive concept. The following drawings and description are to be regarded as illustrative in nature and not restrictive.

### BRIEF DESCRIPTION OF THE DRAWINGS

The present invention and its features will be better understood by reference to the accompanying drawings, wherein:

FIG. 1 is a view of an embodiment of a medical instrument for dislodging, collecting, and removing vertebral disc material fashioned in accordance with the present principles, a vertebral disc material scoop of which is in a fully open position;

FIG. 2 is an enlarged side view of the vertebral disc material dislodger, collector, and remover of FIG. 1 with the vertebral disc material scoop in a fully open position;

FIG. 3 is an enlarged front side view of the collection arm/scoop end of the vertebral disc material dislodger, collector, and remover of FIG. 1 with the vertebral disc material scoop in the fully open position;

FIG. 4 is a rear side view of the vertebral disc material dislodger, collector, and remover of FIG. 1 with the vertebral disc material scoop in the fully open position;

FIG. 5 is a rear side view of the vertebral disc material dislodger, collector, and remover of FIG. 1 with the vertebral disc material scoop in a partially open position;

FIG. 6 is a rear side view of the vertebral disc material dislodger, collector, and remover of FIG. 1 with the vertebral disc material scoop in another partially open position;

FIG. 7 is a rear side view of the vertebral disc material dislodger, collector, and remover of FIG. 1 with the vertebral disc material scoop in a fully closed position;

FIG. 8 is an enlarged side view of the vertebral disc material dislodger, collector, and remover of FIG. 1 with the vertebral disc material scoop in the fully open position;

FIG. 9 is an enlarged side view of the vertebral disc material dislodger, collector, and remover with the vertebral disc material scoop in a partially open position;

FIG. 10 is an enlarged side view of the vertebral disc material dislodger, collector, and remover of FIG. 1 with the vertebral disc material scoop in a partially open position;

FIG. 11 is an enlarged side view of the vertebral disc material dislodger, collector, and remover of FIG. 1 with the vertebral disc material scoop in the fully closed position;

FIG. 12 is an enlarged side view of an end of the vertebral disc material dislodger, collector, and remover of FIG. 1 opposite the vertebral disc material scoop;

FIG. 13 is a view of the vertebral disc material dislodger, collector, and remover of FIG. 1 situated inside and extending from a cannula with the vertebral disc material scoop in a fully open position;

FIG. 14 is a view of the vertebral disc material dislodger, collector, and remover of FIG. 1 situated inside and extending from the cannula with the vertebral disc material scoop in a partially open position;

FIG. 15 is a view of the vertebral disc material dislodger, collector, and remover of FIG. 1 situated inside and extending from the cannula with the vertebral disc material scoop in a partially open position;

FIG. 16 is a view of the vertebral disc material dislodger, collector, and remover of FIG. 1 situated inside and extending from the cannula with the vertebral disc material scoop in a fully closed position;

FIG. 17 is a view of the vertebral disc material dislodger, collector and remover of FIG. 1 situated inside and partially retreating into the cannula with the vertebral disc material scoop in a fully closed position;

FIG. 18 is a view of an embodiment of a vertebral disc material dislodger fashioned in accordance with the present principles, a vertebral disc material scoop of which is in a fully closed position;

FIG. 19 is an enlarged view of the vertebral disc material dislodger of FIG. 18 with the vertebral disc material scoop in a fully closed position;

FIG. 20 is a view of the vertebral disc material dislodger of FIG. 18 with the vertebral disc material scoop in a partially open position;

FIG. 21 is a view of the vertebral disc material dislodger of FIG. 18 with the vertebral disc material scoop in a partially open position;

FIG. 22 is an enlarged sectional view of the vertebral disc material dislodger of FIG. 18 with the vertebral disc material scoop in a fully closed position;

FIG. 23 is an enlarged sectional view of the vertebral disc material dislodger of FIG. 18 with the vertebral disc material scoop in a partially open position;

FIG. 24 is a sectional view of the vertebral disc material dislodger of FIG. 18 with the vertebral disc material scoop in a partially open position;

FIG. 25 is a view of the vertebral disc material dislodger of FIG. 18 situated inside and beginning to extend from a cannula with the vertebral disc material scoop in a fully closed position;

FIG. 26 is a view of the vertebral disc material dislodger of FIG. 18 situated inside and extending from the cannula with the vertebral disc material scoop in a fully closed position; and

FIG. 27 is a view of the vertebral disc material dislodger of FIG. 18 situated inside and extended from the cannula with the vertebral disc material scoop in a partially open position.

For the purposes of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

### DETAILED DESCRIPTION OF THE INVENTION

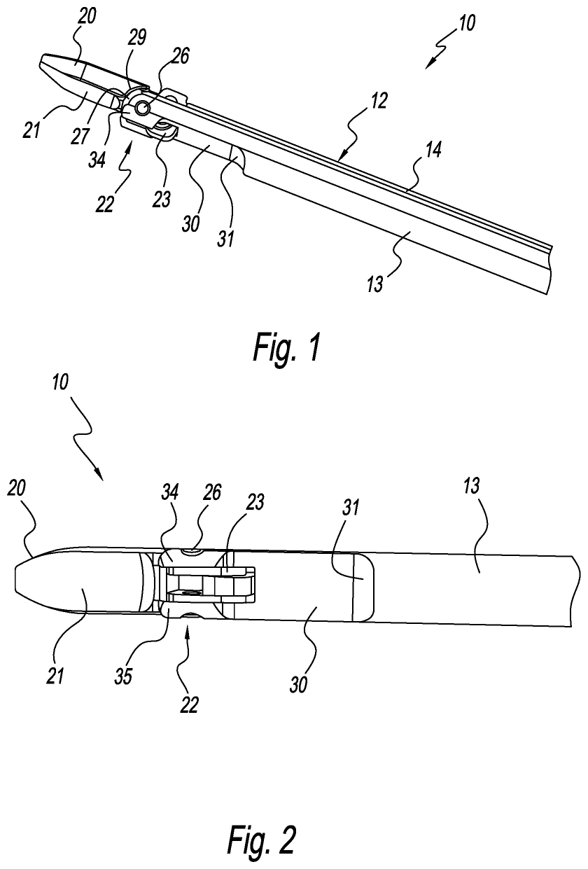

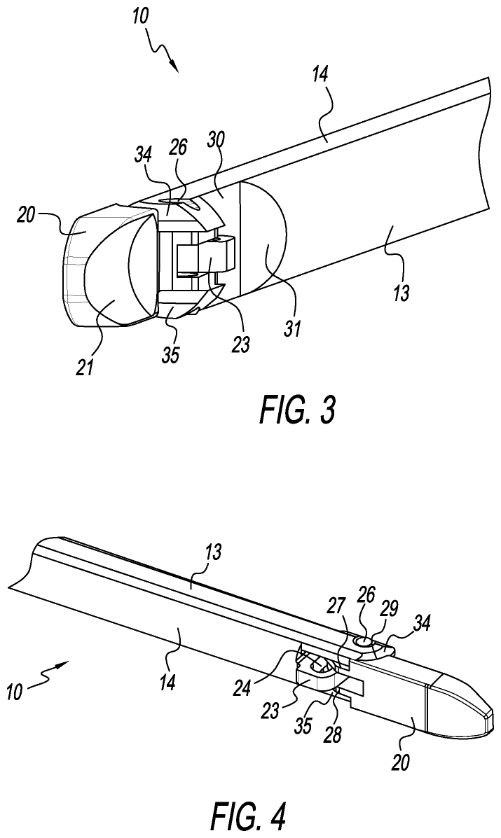

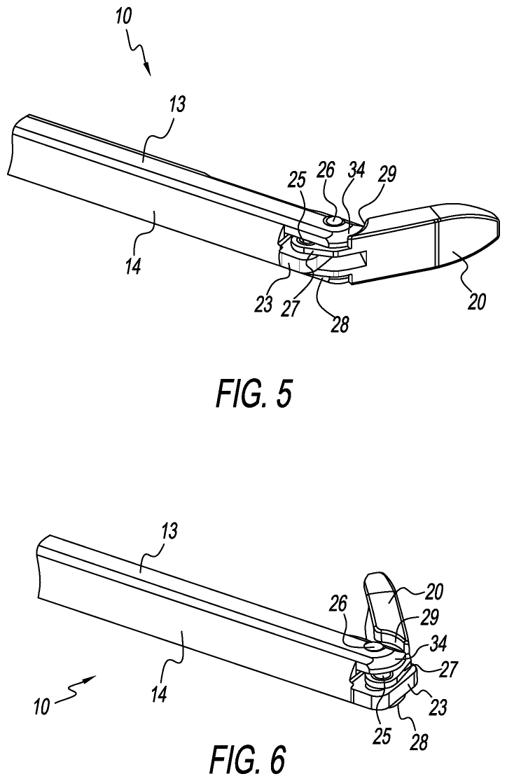

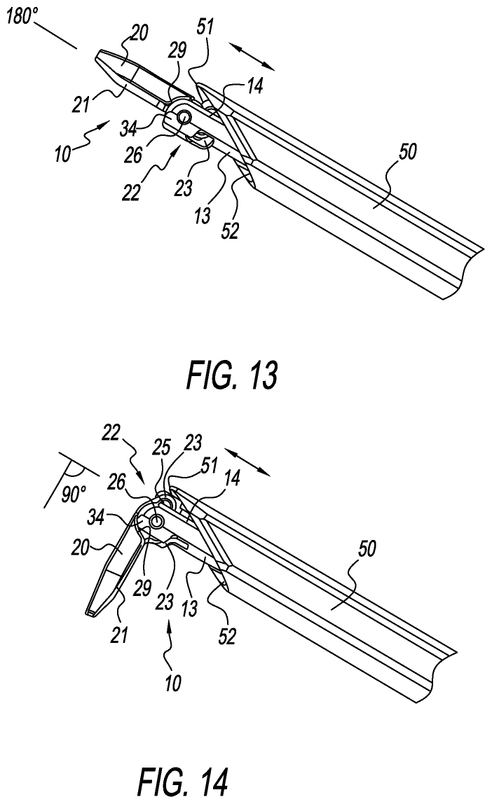

Referring to the FIGS. 1-12, there is shown an embodiment of a medical instrument, generally designated 10 for dislodging, collecting, and removing vertebral disc material (not shown) from a vertebral disc (not shown) in a vertebral disc space (not shown) via a cannula 50 (see FIGS. 13-17) particularly, but not necessarily, in a minimally invasive or micro invasive spine procedure/surgery. The medical instrument 10 is made from one to more surgical grade materials.

In particular, FIGS. 1-12 show various views of the medical instrument 10. In general, the medical instrument 10 has an elongate body 12 that is sized in diameter and length to fit into and be operable via a cannula, endoscope or the like (collectively, cannula). The elongate body 12 has a first or stationary elongate shaft, part, or portion 13 (first or stationary shaft) and a second or movable elongate shaft, part, or portion 14 (second or actuator shaft), the nomenclature first and second being arbitrary. As best seen in FIG. 12, the elongate stationary shaft 13 has a proximal end 15 (opposite its distal end) and a configured tail (distal end) 16 as part of a dovetail joint while the elongate actuator shaft 14 has a proximal end 17 with a configured socket 18 as part of the dovetail joint between the elongate stationary shaft 13 and the elongate actuator shaft 14. The dovetail joint provides positive connection between the elongate stationary shaft 13 and the elongate actuator shaft 14 while allowing easy linear (axial) movement or translation of the elongate actuator shaft 14 relative to the elongate stationary shaft 13.

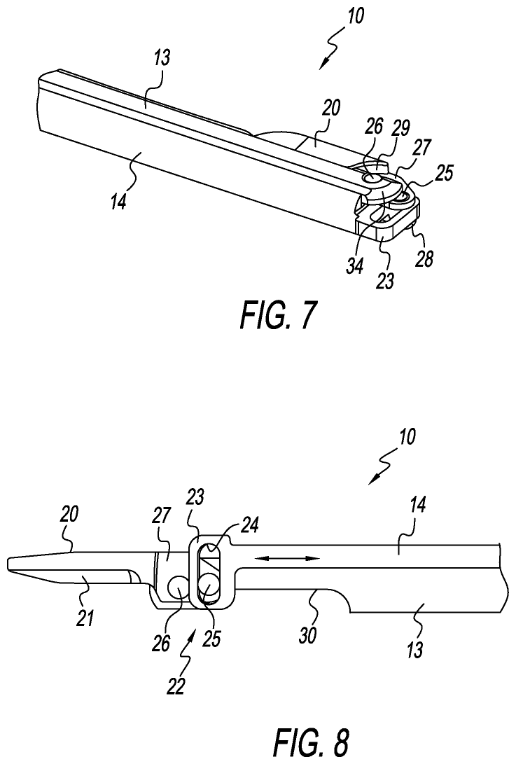

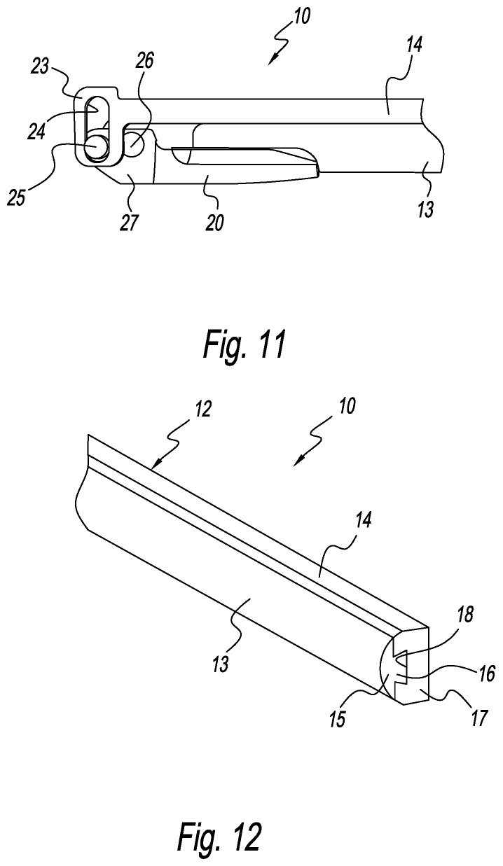

A vertebral disc material scoop or scoop arm (collectively, scoop) 20, is pivotally connected at the distal end of the elongate stationary shaft 13 via a pivot pin 26 that extends from a first curved end 34 on one side of the elongate stationary shaft 13 to a second curved end 35 on another side of the elongate stationary shaft 13 opposite the one side of the elongate stationary shaft 13 and through a first side plate 27 of and on one side of the scoop 20 adjacent the first curved end 34 of the elongate actuator shaft 14, and a second side plate 28 and on the other side of the scoop 20 opposite the first side plate 27 adjacent the second curved end 35 of the elongate stationary shaft 13, the nomenclature first and second being arbitrary. The scoop 20 has a concave surface, cutout, or cavity 21 on its inner surface for receiving (collecting) and holding vertebral disc material (not shown). The scoop 20 is also shaped and configured to cut into (dislodge) and remove (collect) vertebral disc material. In conjunction with the cavity 21 of the scoop 20, the distal end of the elongate stationary shaft 13 has a cutout 30 that aids in keeping or retaining the dislodged vertebral disc material in the scoop 20 and against the medical instrument 10 by creating a pocket in the distal end of the elongate stationary shaft 13 that is shaped to receive the scoop 20. When the scoop 20 is in a closed position (see, e.g., FIGS. 7 and 11) the end of the scoop 20 abuts the end 31 of the elongate stationary shaft 13.

A pivot structure 22 is provided on and between the distal ends of the elongate stationary shaft 13 and the elongate actuator shaft 14 comprising a flange 23 having a slot 24 fixed to the distal end of the elongate actuator shaft 14 and a post 25 extending from the scoop 20 and captured in the slot 24 for motion within the post 25. The scoop 20 pivot pin 26 and associated side plates 27, 28 may also be considered as part of the pivot structure 22. The end 29 of the scoop 20 is curved in like manner as the curved ends 34, 35 to provide guided pivoting of the scoop 20 about the ends 34, 35, among other reasons.

In FIGS. 1-4, the scoop 20 has been pivoted from a closed or initial and ending position (see, e.g., FIG. 7) to a fully open position ready for collecting/dislodging vertebral disc material where a longitudinal axis of the scoop 20 is co-axial with the longitudinal axis of the elongate instrument body 12 of the medical instrument 10. The fully open position is achieved through linear translation of the elongate actuator shaft 14 relative to the elongate stationary shaft 13 along the longitudinal axis of the instrument body 12. This is more fully described below with reference to FIGS. 8-11. The closed position is shown in FIG. 7.

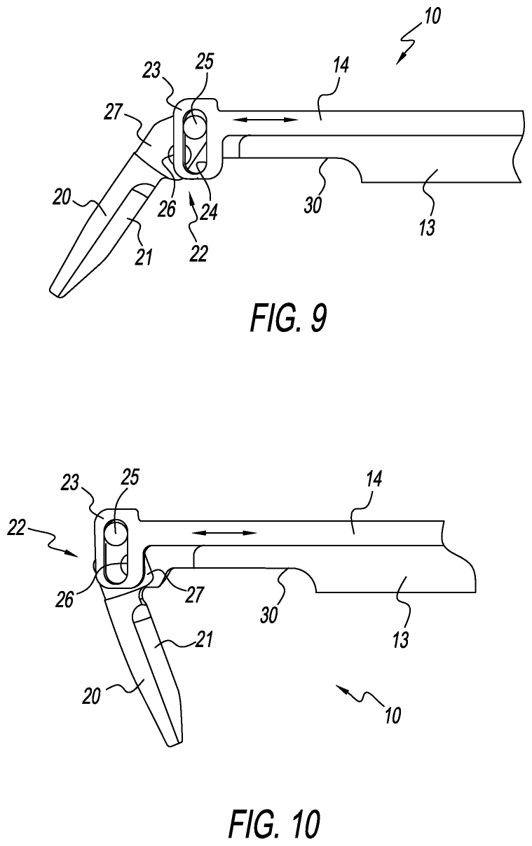

FIGS. 8-11 each depict a side view of the present medical instrument 10 and illustrate the pivoting of the scoop 20 from the fully open or extended position (FIG. 8) where the longitudinal axis of the scoop 20 is co-axial with the instrument body 12 and the scoop 20 is ready to dislodge and collect vertebral disc material. In this position, the scoop 20 is also within the diameter of the instrument body 12 and the post 25 of the scoop 20 has translated through confinement by and within the slot 24 of the flange 23. FIG. 9 depicts an intermediate position of the scoop 20 as the scoop 20 pivots from the fully open position depicted in FIG. 8 by the axial translation of the elongate actuator shaft 14 relative to the elongate stationary shaft 13 and the post 25 of the scoop 20 has translated through confinement by and within the slot 24 of the flange 23. In this position, the scoop 20 is swiping a vertebral disc (not shown) for vertebral disc material dislodging and collection. FIG. 10 depicts another intermediate position of the scoop 20 as the scoop 20 further pivots from the intermediate position depicted in FIG. 9 by further axial translation of the elongate actuator shaft 14 relative to the elongate stationary shaft 13 and the post 25 of the scoop 20 has translated through confinement by and within the slot 24 of the flange 23. In this position, the scoop 20 has collected vertebral disc material from the vertebral disc. FIG. 11 depicts the scoop 20 in the closed position where the collected vertebral disc material is held or captured in the cavity 21 of the scoop 20 and held within the pocket 30 of the elongate stationary shaft 13 for extraction from the patient upon further axial translation of the elongate actuator shaft 14 relative to the elongate stationary shaft 13 and the post 25 of the scoop 20 has translated through confinement by and within the slot 24 of the flange 23.

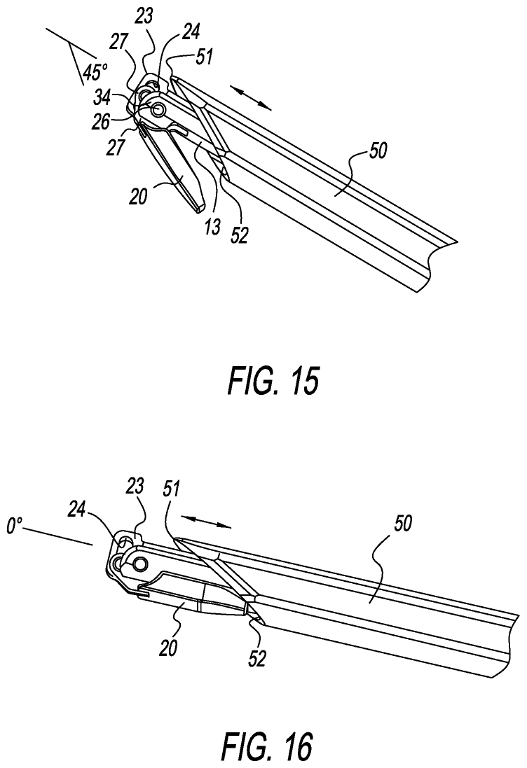

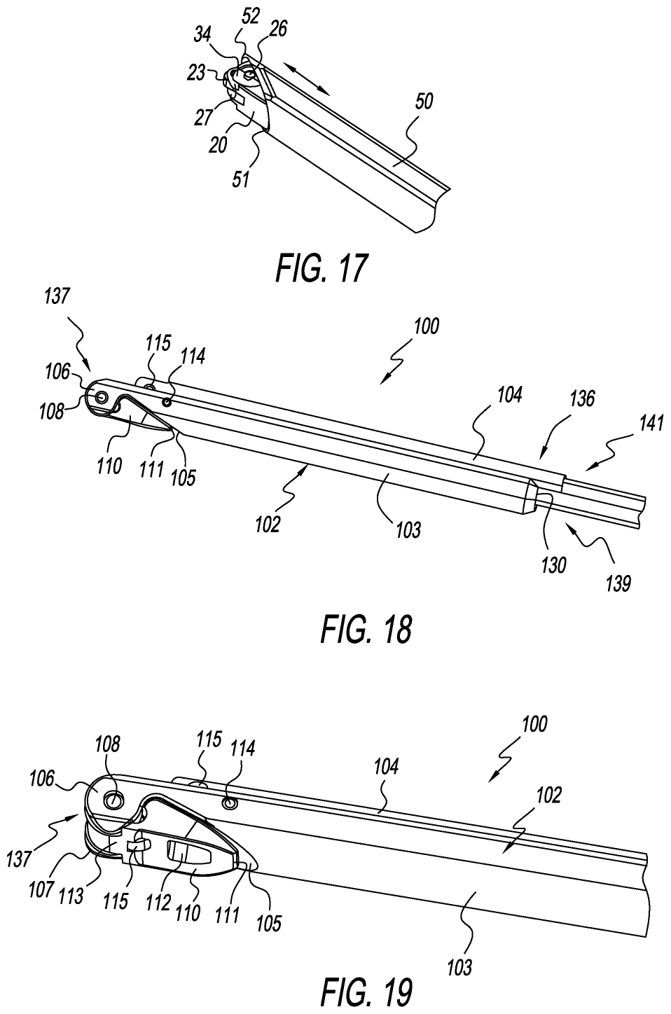

FIGS. 13-17 show the medical instrument 10 within the bore 52 of a cannula 50, the cannula 50 having a slanted end 51 to allow the present medical instrument 10 to fully accomplish its task. FIGS. 13-17 illustrate how the medical instrument 10 dislodges and collects vertebral disc material (not shown) by swiping or scooping against a vertebral disc (not shown). In FIG. 13, the medical instrument 10 has been extended through the cannula 50 such that the scoop 20 is free from the slanted end 51 of the cannula 50 and has been pivoted from a closed position to the fully open position. The fully open position may be considered a 180° position (relative to the longitudinal axis of the instrument body 12 and the cannula 50). In FIG. 14, the scoop 20 has been pivoted by axial translation of the elongate actuator shaft 14 into a 90° position. In FIG. 15, the scoop 20 has been pivoted by axial translation of the elongate actuator shaft 14 into a 45° position. In FIG. 16, the scoop 20 has been pivoted by axial translation of the elongate actuator shaft 14 into a 0° closed position. In FIG. 17, the medical instrument 10 is being drawn back into the cannula 50 for extraction from the cannula 50 and the patient's body (not shown). The captured vertebral disc material is thus removed or extracted from the patient's body.

A method of obtaining vertebral disc material from a vertebral disc space using the medical instrument 10 includes making an incision in a patient's body (not shown) proximate to the vertebral disc, inserting a cannula 50 into the patient's body through the incision such that an end of the cannula 50 is proximate the vertebral disc, inserting the medical instrument 10 into the cannula 50 such that the distal end of the medical instrument 10 extends beyond the end of the cannula 50, axially translating the elongate actuator shaft 14 in a first axial direction relative to the elongate stationary shaft 13 to pivot the vertebral disc material scoop 20 from a 0° position to a 180° position, axially translating the elongate actuator shaft 14 relative to the elongate stationary shaft 13 in a second axial direction opposite the first axial direction to pivot the vertebral disc material scoop 20 from the 180° position to the 0° position to dislodge and collect vertebral disc material from the vertebral disc, and removing the medical instrument 10 from the cannula to remove the dislodged vertebral disc material from the patient's body.

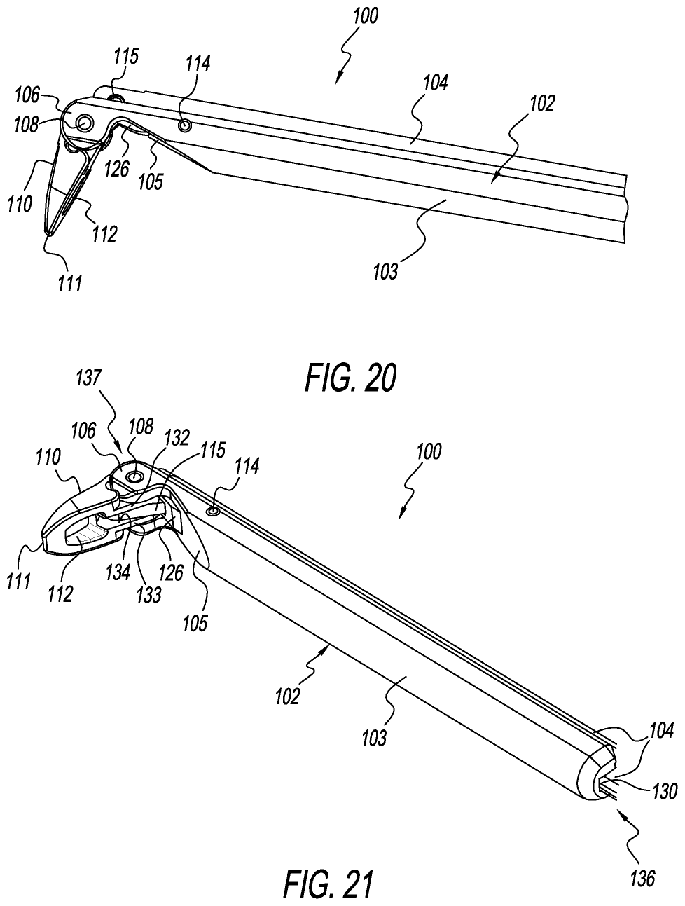

Referring to FIGS. 18-24, there is shown an embodiment of a medical instrument, generally designated 100 for dislodging (dislodger 100) vertebral disc material (not shown) from a vertebral disc (not shown) in a vertebral disc space (not shown) via a cannula 50 (see FIGS. 25-27) particularly, but not necessarily, in a minimally invasive or micro invasive spine procedure/surgery. The medical instrument 100 is made from one to more surgical grade materials.

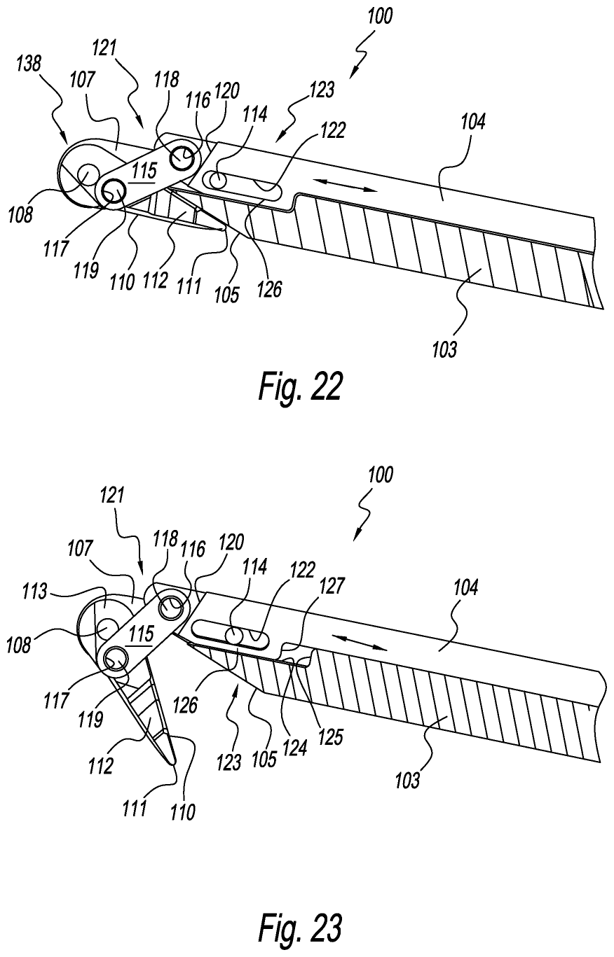

FIGS. 18-24 show various views of the medical instrument 100. In general, the medical instrument 100 has an elongate body/body structure 102 with a proximal end 136 (see FIG. 21) and a distal end 137 (see FIG. 19) that is sized in diameter and length to fit into and be operable through the cannula 50 (see FIGS. 25-27). The elongate body structure 102 has a first or stationary elongate shaft, body, or portion (collectively, first or stationary elongate shaft) 103 and a second or movable elongate shaft, body, or portion (collectively, second, movable, or actuator shaft) 104 that serves, at least in part, as an actuator, the nomenclature first and second being arbitrary. A pivoting scoop structure 121 having a scoop or scoop arm 110 (see FIGS. 22 and 23) defining a nose 111, is situated on the distal end 137 of the elongate body structure 102. The movable elongate shaft 104 may be considered an actuator for the pivoting scoop structure 121/scoop 110. As best seen in FIGS. 19-21, a distal end 138 of the first elongate shaft 103 has a slant or angle 105 that is configured to accommodate the scoop 110 when the scoop 110 is in a fully closed or 0° position (FIG. 19) such that the medical instrument 100 may fit through a cannula.

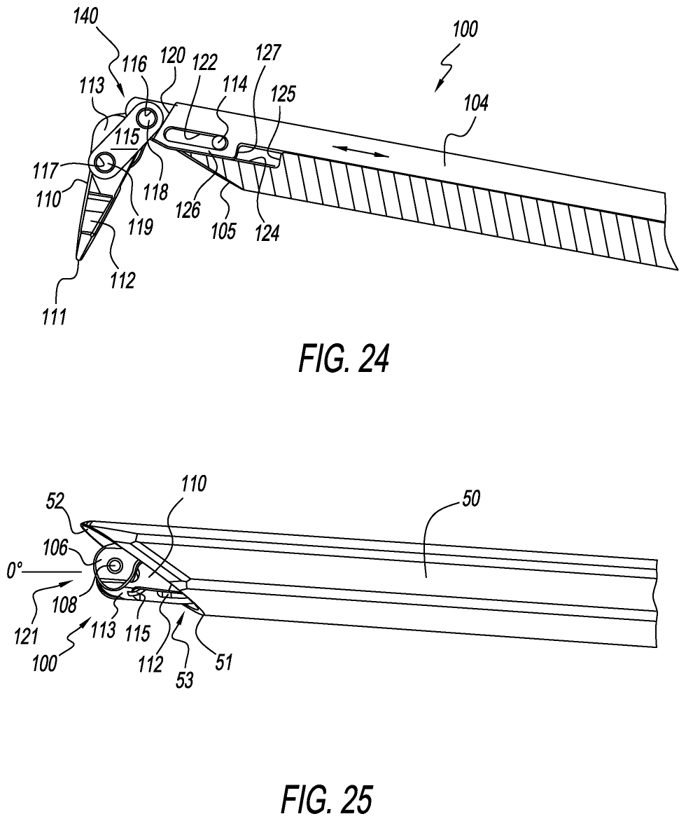

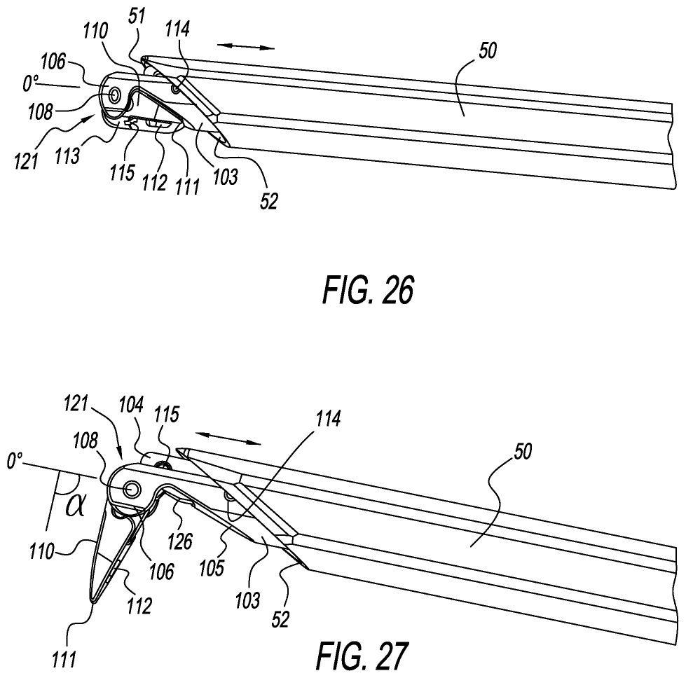

With reference to FIGS. 18-24 particulars of the medical instrument 100 will now be described. The first elongate shaft 103 has a proximal end 139 (see e.g., FIG. 18), the distal end 138 (see e.g., FIG. 22), and an elongate slot 130 that extends from the proximal end 139 of the first elongate shaft 103 (the proximal first elongate shaft end 139) to the distal end 138 of the first elongate shaft 103 (the distal first elongate shaft end 138). The second elongate shaft 104 is situated in the elongate slot 130 of the first elongate shaft 103 and movable along its longitudinal axis in proximal to distal, and distal to proximal directions. Axial travel of the second elongate shaft 104 within the elongate slot 130 however, is limited in distance in the distal to proximal direction by structure. The second elongate shaft 104 defines a proximal end 141 (see e.g., FIG. 18), and a distal end 140 (see e.g., FIG. 24). As best seen in FIGS. 22-24, a notch 124 is formed in the first elongate shaft 103. Particularly, the notch 124 is situated in the distal first elongate shaft end 138, and axially extends therefrom to an axial length towards the proximal first elongate shaft end 139, then terminates in a wall or vertical portion 125. The wall or vertical portion 125 defines a stop for an end 127 of a flange 126 that is situated on the second elongate shaft 104 proximate the distal second elongate shaft end 140 (see e.g., FIGS. 23, 24), and thus defines an axial translation stop for the second elongate shaft 104 relative to the first elongate shaft 103. The length of travel of the second elongate shaft 104 relative to the first elongate shaft 103 corresponds to the arcuate travel distance of the scoop 110 from 0° to 180°, or vice versa. Direction of axial translation of the second elongate shaft 104 relative to the first elongate shaft 103 determines whether the scoop 110 is opening (from a 0° position—see FIG. 26—through a 180° position—while a 180° position is not shown for the medical instrument 100, the 180° position of the scoop 110 would be the same as the 180° position of the scoop 20 of the medical instrument 10 of FIG. 13), or closing/swiping/scooping (from the 180° position through the 0° position). Axial translation of the second elongate shaft 104 in the proximal direction closes the scoop 110 from an open position to a closed position, while axial translation of the second elongate shaft 104 in the distal direction opens the scoop 110 from the closed position to an open position.

Returning to FIGS. 18-24, the distal first elongate shaft end 138 is bifurcated, defining a first arm 106 and a second arm 107 situated opposite the first arm 106. A rounded scoop end 113 of the scoop 110 is disposed between the first and second arms 106, 107 with a pivot pin 108 extending through the first arm 106, the rounded scoop end 113, and the second arm 107 such that the scoop 110 is pivotally retained between the first and second arms 106, 107. The scoop 110 also has an opening or window 112 that extends through the scoop from one side thereof to the opposite side thereof. The opening 112 both aids in dislodging vertebral disc material that is outside of the perimeter of the MIS tube (cannula) the medical instrument 100 it is inserted through, and to aid in evacuating the dislodged vertebral disc material when the scoop is fully closed or collapsed. Should the dislodged vertebral disc material not be able to escape the scoop/scoop area, the greater chance the scoop would not be able to fully close, possible hindering removal of the medial instrument 100 from the cannula. The pivoting scoop 110 is further connected to a pivoting scoop structure 121 that provides linkage between the second elongate shaft 104 and the scoop 110 to aid in effecting motion to pivot the scoop 110 about the first and second arms 106, 107 of the bifurcated distal first elongate shaft end 138 of the first elongate shaft 103 through axial translation of the second elongate shaft 104.

The pivoting scoop structure 121 includes a plate or connection arm 115 having a first bore 116 at a first rounded end thereof, and a second bore 117 at a second rounded end thereof, the nomenclature first and second being arbitrary. A first connection pin 118 is disposed in the first bore 116 of the connection arm 115 and through the distal second elongate shaft end 140, such that the connection arm 115 can swivel about the distal second elongate shaft end 140. A second connection pin 119 is disposed in the second bore 117 of the connection arm 115 and through the scoop 110, such that the connection arm 115 can effect swiveling of the scoop 110 about pivot 108, and swiveling of the connection arm 115 about the second connection pin/pivot 119. Axial movement of the second elongate shaft 104 effects motion of the connection arm 115 to effect motion of the scoop 110. As best seen in FIG. 21, the connection arm 115 extends out from a collar 126 of the first elongate shaft 103 and is attached via second connection pin 119 to a bifurcated scoop structure comprising a first leg 132 and a second leg 133, the nomenclature first and second being arbitrary. The second end of the connection is situated between the first and second legs 132, 133 with pivot pin 108 providing a connection point.

FIGS. 22-23 illustrate the manner in which longitudinally axial translation of the second elongate shaft 104 on the first elongate shaft 103 (denoted by the two-headed arrows) effects pivoting of the scoop 110 for swiping through its vertebral disc material dislodging arc (0° to 180°). FIG. 22 depicts the scoop 110 in a fully closed (0°) position with the second elongate shaft 104 fully axially translated in the proximal direction such that the end 127 of the flange 126 thereof abuts the wall 125 of the notch 124 of the first elongate shaft 104. The translation pin 114 of the first elongate shaft 103 is to the distal side (end) in the horizontal slot 122 of the flange 126 of the second elongate shaft 104. This may be considered a beginning or an ending position. FIG. 23 depicts the scoop 110 in a partially open position (˜60°) with the second elongate shaft 104 partially axially translated in the distal direction such that the end 127 of the flange 126 is distally away from the wall 125. The translation pin 114 of the first elongate shaft 103 is within the horizontal slot 122 of the flange 126 of the second elongate shaft 104 between the distal and proximal ends of the slot 122. This may be considered a partially open position (or a mostly closed position). FIG. 24 depicts the scoop 110 in a continued partially open position (˜105°) with the second elongate shaft 104 almost fully axially translated in the distal direction such that the end 127 of the flange 126 is almost fully opposite the wall 125. The translation pin 114 of the first elongate shaft 103 is within the horizontal slot 122 almost abutting the proximal end of the horizontal slot 122. This may be considered a mostly open position (or a partially closed position).

FIGS. 25-27 show the medical instrument 100 within the bore 52 of a cannula 50, the cannula 50 having a slanted end 51 to allow the present medical instrument 100 to fully accomplish its task. FIGS. 25-27 illustrate how the medical instrument 100 operates to dislodge vertebral disc material by swiping/swiveling the scoop 110 against a vertebral disc (not shown). The double-headed arrow in the Figures denotes axial movement of the medical instrument 100 in the cannula 50. In FIG. 25, the medical instrument 100 has been extended through the cannula 50 from the proximal end of the cannula (not seen) to its distal end 53 such that the pivoting scoop structure 121 with its scoop 110 is emerging from the bore 52 of the cannula 50. In FIG. 26, the medical instrument 100 has been axially moved such that the head of the medical instrument 100 with its pivot structure 121 has fully emerged from the bore 52 of the cannula 50. The scoop 110 is in a fully closed position. In FIG. 27, the medical instrument 100 has been axially moved more such that the head of the medical instrument 100 with its pivot structure 121 has more fully emerged from the bore 52 of the cannula 50. The scoop 110 has been pivoted into a partially open position (at an angle α relative to the longitudinal axis—denoted 0°).

A method of obtaining vertebral disc material from a vertebral disc space using the medical instrument 100 includes making an incision in a patient's body (not shown) proximate to a vertebral disc whose vertebral disc material is to be dislodged, inserting a cannula 50 into the patient's body through the incision such that a distal end 53 of the cannula 50 is proximate the vertebral disc, inserting the medical instrument 100 into the cannula 50 such that the scoop 110 and scoop pivot structure 121 (collectively, head or distal end) of the medical instrument 100 extends beyond the end 53 of the cannula 50, axially translating the second elongate shaft 104 in a first axial direction relative to the first elongate shaft 103 to pivot the scoop 110 from, through, and between 0° to 180°, axially translating the second actuator shaft 104 relative to the first elongate shaft 103 in a second axial direction opposite the first axial direction to pivot the scoop 110 from 180° through and to 0° to dislodge vertebral disc material from the vertebral disc, and removing the medical instrument 100 from the cannula to allow removal of the dislodged vertebral disc material from the patient's body via a separate medical instrument (e.g. a rongeur).

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention.

1. A medical instrument for dislodging vertebral disc material from a vertebral disc in a vertebral disc space comprising:
an elongate body defining a longitudinal axis, an elongate body proximal end, and an elongate body distal end, the elongate body characterized by a first elongate shaft defining a first elongate shaft proximal end, and a bifurcated first elongate shaft distal end, and a second elongate shaft defining a second elongate shaft proximal end, and a bifurcated second elongate shaft distal end, the second elongate shaft axially movably situated on the first elongate shaft along the longitudinal axis of the elongate body;
a scoop defining a proximal scoop end with a slot, a distal scoop end, and pivotally connected to the bifurcated first elongate shaft distal end via a pivot pin;
a pivot structure having a first portion and a second portion, the first portion of the pivot structure received in the slot of the proximal scoop end and pivotally coupled to the proximal scoop end via a first pivot structure pin, with the second portion of the pivot structure pivotally coupled to the bifurcated distal second elongate shaft end of the second elongate shaft via a second pivot structure pin; and
an actuator structure of the second elongate shaft situated proximate the bifurcated second elongate shaft distal end and the second portion of the pivot structure retains an actuator pin of the first elongate shaft within an actuator slot of the actuator structure with the actuator pin providing a guide therein for the actuator structure such that axial translation of the second elongate shaft on the first elongate shaft along the longitudinal axis of the elongate body causes the pivot structure to swivel effecting pivoting of the scoop about the first portion of the pivot structure and the bifurcated first elongate shaft distal end from, through, and between 0° to 180° relative to the longitudinal axis of the elongate body, the swiveling motion of the scoop dislodging vertebral disc material from the vertebral disc outside of the perimeter of a cannula.

2. The medical instrument of claim 1, wherein the scoop has a cutout extending from one side of the scoop to another side of the scoop opposite the one side of the scoop.

3. The medical instrument of claim 2, wherein the one side of the scoop is angled from the distal scoop end to the proximal scoop end, and the first elongate shaft includes an angle proximate to the bifurcated first elongate shaft distal end that is complementary to the angle of the one side of the scoop.

4. The medical instrument of claim 3, wherein the distal scoop end is rounded.

5. The medical instrument of claim 1, wherein the actuator slot is disposed along the longitudinal axis of the elongate body.

6. A method for dislodging vertebral disc material from a vertebral disc in a vertebral disc space via spine surgery using a cannula, the method comprising:
accessing a vertebral disc space of a patient via a cannula;
inserting a medical instrument through the cannula for dislodging vertebral disc material from a vertebral disc in a vertebral disc space, the medical instrument having:
an elongate body defining a longitudinal axis, an elongate body proximal end, and an elongate body distal end, the elongate body characterized by a first elongate shaft defining a first elongate shaft proximal end, and a bifurcated first elongate shaft distal end, and a second elongate shaft defining a second elongate shaft proximal end, and a bifurcated second elongate shaft distal end, the second elongate shaft axially movably situated on the first elongate shaft along the longitudinal axis of the elongate body;
a scoop defining a proximal scoop end with a slot, a distal scoop end, and pivotally connected to the bifurcated first elongate shaft distal end via a pivot pin;
a pivot structure having a first portion and a second portion, the first portion of the pivot structure received in the slot of the proximal scoop end and pivotally coupled to the proximal scoop end via a first pivot structure pin, with the second portion of the pivot structure pivotally coupled to the bifurcated distal second elongate shaft end of the second elongate shaft via a second pivot structure pin; and
an actuator structure of the second elongate shaft situated proximate the bifurcated second elongate shaft distal end and the second portion of the pivot structure retains an actuator pin of the first elongate shaft within an actuator slot of the actuator structure with the actuator pin providing a guide therein for the actuator structure such that axial translation of the second elongate shaft on the first elongate shaft along the longitudinal axis of the elongate body causes the pivot structure to swivel effecting pivoting of the scoop about the first portion of the pivot structure and the bifurcated first elongate shaft distal end from, through, and between 0° to 180° relative to the longitudinal axis of the elongate body, the swiveling motion of the scoop dislodging vertebral disc material from the vertebral disc outside of the perimeter of a cannula;
axially translating the second elongate shaft in a first axial direction relative to the first elongate shaft to pivot the scoop from a 0° position to a 180° position;
axially translating the second elongate shaft relative to the first elongate shaft in a second axial direction opposite the first axial direction to pivot the scoop from the 180° position to the 0° position outside of the axial plane and beyond the perimeter of the cannula to dislodge vertebral disc material from the vertebral disc; and
removing the medical instrument from the cannula.

7. The method of claim 6, wherein the scoop has a cutout extending from one side of the scoop to another side of the scoop opposite the one side of the scoop.

8. The method of claim 7, wherein the one side of the scoop is angled from the distal scoop end to the proximal scoop end, and the first elongate shaft includes an angle proximate to the bifurcated first elongate shaft distal end that is complementary to the angle of the one side of the scoop.

9. The method of claim 8, wherein the actuator slot is disposed along the longitudinal axis of the elongate body.

10. A medical instrument for dislodging, collecting and removing vertebral disc material from a vertebral disc in a vertebral disc space comprising:
an elongate body defining a longitudinal axis, an elongate body distal end, and an elongate body proximal end, the elongate body characterized by an elongate stationary shaft and an elongate actuator shaft axially movably connected to the elongate stationary shaft along the longitudinal axis of the elongate body, the elongate stationary shaft defining an elongate stationary shaft distal end, and an elongate stationary shaft proximal end, and the elongate actuator shaft defining an elongate actuator shaft distal end and an elongate actuator shaft proximal end;
a vertebral disc material collector having a post at one end thereof and pivotally connected to the elongate actuator shaft distal end, the vertebral disc material collector configured to dislodge, collect, and hold dislodged vertebral disc material; and
a pivot structure situated at the elongate stationary shaft distal end for receiving the post of the vertebral disc material collector and providing pivoting of the vertebral disc material collector about the post from a 0° position to a 180° position relative to the longitudinal axis of the elongate body through axial translation of the elongate actuator shaft relative to the elongate stationary shaft in a first axial direction and from the 180° position to the 0° position through axial translation of the elongate actuator shaft relative to the elongate stationary shaft in a second axial direction opposite the first axial direction such that the vertebral disc material collector extends beyond a perimeter of a cannula to obtain vertebral disc material from the vertebral disc.

11. The medical instrument of claim 10, wherein the elongate actuator shaft is axially movably connected to the elongate stationary shaft via a dovetail connection.

12. The medical instrument of claim 11, wherein the vertebral disc material collector is fashioned as a scoop with a cavity for collecting dislodged vertebral disc material.

13. The medical instrument of claim 12, further comprising:
a pocket formed in the actuator shaft distal end configured to receive the scoop and retain vertebral disc material in the cavity of the scoop when the scoop is in a fully closed position.

14. The medical instrument of claim 10, wherein the pivot structure comprises a flange with a slot, the slot receiving the post of the vertebral disc material collector such that movement of the elongate actuator shaft causes the post to translate in the slot to provide pivoting of the vertebral disc material collector from 0° to 180° and from 180° to 0°.

15. The medical instrument of claim 14, wherein the slot comprises a vertical slot.

16. A method for dislodging, collecting, and removing vertebral disc material from a vertebral disc in a vertebral disc space via spine surgery using a cannula, the method comprising:
accessing a vertebral disc space of a patient via a cannula;
inserting a medical instrument through the cannula for dislodging, collecting, and removing vertebral disc material from a vertebral disc in a vertebral disc space, the medical instrument having:
an elongate body defining a longitudinal axis, an elongate body distal end, and an elongate body proximal end, the elongate body characterized by an elongate stationary shaft and an elongate actuator shaft axially movably connected to the elongate stationary shaft along the longitudinal axis of the elongate body, the elongate stationary shaft defining an elongate stationary shaft distal end, and an elongate stationary shaft proximal end, and the elongate actuator shaft defining an elongate actuator shaft distal end and an elongate actuator shaft proximal end;
a vertebral disc material collector having a post at one end thereof and pivotally connected to the elongate actuator shaft distal end, the vertebral disc material collector configured to dislodge, collect, and hold dislodged vertebral disc material; and
a pivot structure situated at the elongate stationary shaft distal end for receiving the post of the vertebral disc material collector and providing pivoting of the vertebral disc material collector about the post from a 0° position to a 180° position relative to the longitudinal axis of the elongate body through axial translation of the elongate actuator shaft relative to the elongate stationary shaft in a first axial direction and from the 180° position to the 0° position through axial translation of the elongate actuator shaft relative to the elongate stationary shaft in a second axial direction opposite the first axial direction such that the vertebral disc material collector extends beyond a perimeter of a cannula to obtain vertebral disc material from the vertebral disc;
axially translating the elongate actuator shaft in a first axial direction relative to the elongate stationary shaft to pivot the vertebral disc material collector from a 0° position to a 180° position;
axially translating the elongate actuator shaft relative to the elongate stationary shaft in a second axial direction opposite the first axial direction to pivot the vertebral disc material collector from the 180° position to the 0° position outside of the axial plane and beyond the perimeter of the cannula to collect vertebral disc material from the vertebral disc; and
removing the medical instrument from the cannula.

17. The method of claim 16, wherein the elongate actuator shaft is axially movably connected to the elongate stationary shaft via a dovetail connection.

18. The method of claim 17, wherein the vertebral disc material collector is fashioned as a scoop with a cavity for collection of dislodged vertebral disc material.

19. The method of claim 18, wherein a pocket is formed in the elongated actuator shaft distal end that is configured to receive the scoop and retain dislodged vertebral disc material in the scoop cavity when the scoop is in the 0° position.

20. The method of claim 16, wherein the pivot structure comprises a flange with a vertical slot, the vertical slot receiving the post of the vertebral disc material collector such that movement of the elongate actuator shaft causes the post to translate in the vertical slot of the flange to provide pivoting of the vertebral disc material collector from 0° to 180° and from 180° to 0°.
